(19) United States
(12) Patent Application Publication
(10) Pub. No.: US 2008/0015385 A1
(43) Pub. Date: Jan. 17, 2008

(54) Preparation of (S)-pregabalin-nitrile

(76) Inventor: Lilach Hedvati, Doar Ha Hefer (IL)

Correspondence Address: KENYON & KENYON LLP, ONE BROADWAY, NEW YORK, NY, 10004, US

(21) Appl. No.: 11/809,427

(22) Filed: May 31, 2007

Related U.S. Application Data
(60) Provisional application No. 60/815,611, filed on Jun. 20, 2006.
(60) Provisional application No. 60/831,590, filed on Jul. 17, 2006.
(60) Provisional application No. 60/836,731, filed on Aug. 9, 2006.
(60) Provisional application No. 60/809,978, filed on May 31, 2006.
(60) Provisional application No. 60/831,591, filed on Jul. 17, 2006.
(60) Provisional application No. 60/836,730, filed on Aug. 9, 2006.
(60) Provisional application No. 60/860,360, filed on Nov. 20, 2006.
(60) Provisional application No. 60/879,870, filed on Jan. 10, 2007.
(60) Provisional application No. 60/919,201, filed on Mar. 20, 2007.
(60) Provisional application No. 60/926,059, filed on Apr. 23, 2007.

(51) Int. Cl. C07C 253/00; C07C 205/02

(52) U.S. Cl. 562/553; 558/440

(57) ABSTRACT

Provided are processes for the preparation of (3S)-cyano-5-methylhexanoic acid, an intermediate in the synthesis of (S)-pregabalin.

### CROSS-REFERENCE TO RELATED APPLICATIONS

This application claims the benefit of priority to U.S. provisional application Ser. Nos. 60/815,611, filed Jun. 20, 2006; 60/831,590, filed Jul. 17, 2006; 60/836,731, filed Aug. 9, 2006; 60/809,978, filed May 31, 2006; 60/831,591, filed Jul. 17, 2006; 60/836,730, filed Aug. 9, 2006; 60/860,360, filed Nov. 20, 2006; 60/879,870, filed Jan. 10, 2007; 60/919,201, filed Mar. 20, 2007; and 60/926,059 filed Apr. 23, 2007, hereby incorporated by reference.

### FIELD OF THE INVENTION

The invention encompasses the preparation of (3S)-cyano-5-methylhexanoic acid, an intermediate in the synthesis of (S)-pregabalin.

### BACKGROUND OF THE INVENTION

(S)-Pregabalin, (S)-(+)-3-(aminomethyl)-5-methylhexanoic acid, a compound having the chemical structure, 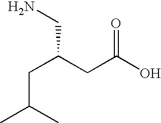
is also known as γ-amino butyric acid or (S)-3-isobutyl GABA. (S)-Pregabalin has been found to activate GAD (L-glutamic acid decarboxylase). (S)-Pregabalin has a dose dependent protective effect on-seizure, and is a CNS-active compound. (S)-Pregabalin is useful in anticonvulsant therapy, due to its activation of GAD, promoting the production of GABA, one of the brain's major inhibitory neurotransmitters, which is released at 30 percent of the brains synapses. (S)-Pregabalin has analgesic, anticonvulsant, and anxiolytic activity.

Processes for the non-asymmetric synthesis of (S)-pregabalin are disclosed in U.S. Pat. No. 5,616,793, Drugs of the Future, 24(8): 862-870 (1999), Synthesis, 955 (1989), and in J. Am. Chem. Soc., 126: 9906 (2004).

U.S. Pat. No. 5,637,767 (“'767 patent”) refers to the preparation of pregabalin through the pregabalin intermediate (±)-3-cyano-5-methylhexanoic acid (“pregabalin nitrile racemate” or “PRG-nitrile racemate”) by decarboxylation of product II, followed by hydrolysis. Then, the pregabalin nitrile racemate is hydrogenated to obtain pregabalin racemate (“PRG-racemate”), followed by optical resolution to obtain (S)-pregabalin. This process may be illustrated by the following Scheme 1. 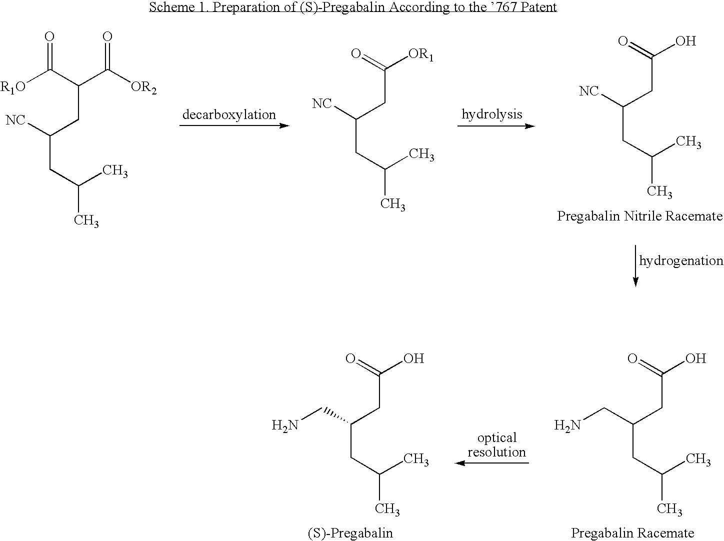
wherein R₁ and R₂ are the same or different and are hydrogen, C₁-C₆ alkyl, aryl, benzyl, or C₃-C₆ cycloalkyl. See, e.g., '767 patent, col. 7, 1. 38 to col. 9, 1. 65; col. 3, 11. 41-43.

The '767 patent states that the optical resolution may be performed by selective crystallization with the (S)-mandelic acid. Id. at col. 9, 11. 17-45. The pregabalin racemate is combined with the (S)-mandelic acid to form a diastereomeric mandelic acid salt of pregabalin. The (S, S) mandelic acid salt of pregabalin is then selectively crystallized, while the (R, S) salt stays in solution. Id. (S)-mandelic acid is then removed from the (S, S) salt to give (S)-pregabalin. Id.

Optical resolution of a racemic mixture via a diastereomeric salt, such as the method referred to in the '767 patent, may be depicted by the following Scheme 2. 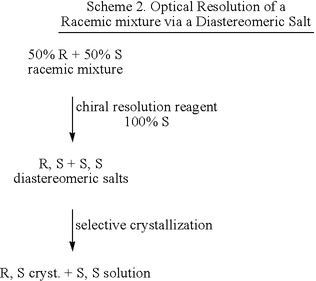
The salt is formed by reacting the racemic mixture with a chiral resolution reagent. Then, a selective crystallization of only one of the diastereomers is done to isolate the desired diastereomer salt, while the undesired diastereomer remains in the solution. The crystalline salt is then isolated, and the chiral resolution reagent is removed to give the desired enantiomer.

Preparing (S)-pregabalin by optically resolving pregabalin racemate, as in the '767 patent, presents the challenge of recycling the unwanted (R)-pregabalin that is also prepared. Because recycling (R)-pregabalin is difficult, such a process would be inefficient and expensive to use on an industrial scale.

Accordingly, there is a need in the art for a process for preparing (S)-pregabalin that does not suffer from the above-described shortcomings of the prior art.

### SUMMARY OF THE INVENTION

In one embodiment, the invention encompasses a process for preparing (3S)-cyano-5-methylhexanoic acid comprising: a) combining a (±)-2-carboxyalkyl-3-cyano-5-methyl hexanoic acid ester of the following structure, 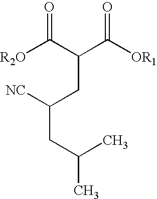
a solvent selected from the group consisting of water, a C₁-C₆ alcohol, and mixtures thereof, and an alkali metal base, to obtain an alkaline salt of pregabalin nitrile of the following structure 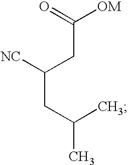
b) combining the above alkaline salt of pregabalin nitrile and an inorganic acid to obtain a mixture having (±)-3-cyano-5-methylhexanoic acid; c) combining the (±)-3-cyano-5-methylhexanoic acid, a solvent selected from the group consisting of ketones, esters, nitrites, C₁₋₄ alcohols, water, and mixtures thereof, and a chiral resolution reagent selected from the group consisting of phenylethylamine, naphtylethylaamine, D-glucamine, L-lysine, L-proline, brucine, sparteine, ephedrine, norephedrine, and salts thereof to obtain a precipitate of a diastereomeric salt; and d) combining the precipitated diastereomeric salt with an inorganic acid to obtain (3S)-cyano-5-methylhexanoic acid, wherein M is an alkali metal and R₁ and R₂ are the same or different and are C₁-C₆ alkyl, aryl, aralkyl, or C₃-C₆ cycloalkyl.

In another embodiment, the invention encompasses a process for optically resolving (3S)-cyano-5-methylhexanoic acid from (±)-3-cyano-5-methylhexanoic acid comprising: a) combining (±)-3-cyano-5-methylhexanoic acid, a solvent selected from the group consisting of ketones, esters, nitrites, C₁₋₄ alcohols, water, and mixtures thereof, and a chiral resolution reagent selected from the group consisting of phenylethylamine, naphtylethylamine, D-glucamine, L-lysine, L-proline, brucine, sparteine, ephedrine, norephedrine, and salts thereof, to obtain a precipitate of a diastereomeric salt; and b) combining the precipitated diastereomeric salt with an inorganic acid to obtain (3S)-cyano-5-methylhexanoic acid.

In another embodiment, the invention encompasses a process for optically resolving (3S)-cyano-5-methylhexanoic acid from (±)-3-cyano-5-methylhexanoic acid comprising: a) providing (±)-3-cyano-5-methylhexanoic acid and b) resolving (3S)-cyano-5-methylhexanoic acid from the (±)-3-cyano-5-methylhexanoic acid with a chiral resolution reagent.

In another embodiment, the invention encompasses a process for preparing (S)-pregabalin comprising preparing (3S)-cyano-5-methylhexanoic acid by any of the above-described processes and converting the (3S)-cyano-5-methylhexanoic acid into (S)-pregabalin.

### DETAILED DESCRIPTION OF THE INVENTION

The invention addresses the above-described shortcomings of the prior art by providing a process for preparing (S)-pregabalin directly from the chiral intermediate (3S)-cyano-5-methylhexanoic acid, thereby avoiding the step of optically resolving pregabalin racemate. The chiral intermediate (3S)-cyano-5-methylhexanoic acid is prepared by optically resolving (±)-3-cyano-5-methylhexanoic acid via a diastereomeric salt using a chiral amine resolution reagent as depicted below.
3-Cyano-5-methylhexanoic acid+(S)-Chiral amine→(S)-PRG-nitrile: (S)-amine salt
3-Cyano-5-methylhexanoic acid+(R)-Chiral amine→(S)-PRG-nitrile: (R)-amine salt

The invention provides a process for preparing (S)-pregabalin by optically resolving the intermediate pregabalin nitrile to form (3S)-cyano-5-methylhexanoic acid (“(S)-pregabalin nitrile” or “(S)-PRG-nitrile”), as illustrated in the following Scheme 3. 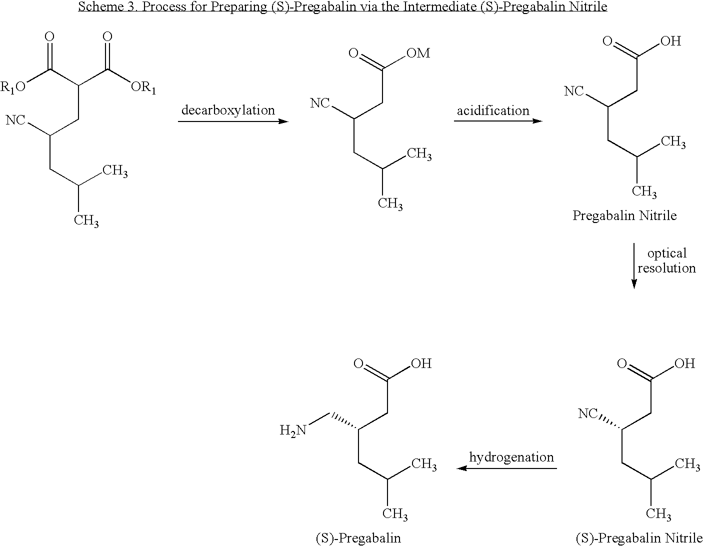
wherein M is an alkali metal and R₁ and R₂ are the same or different and are C₁-C₆ alkyl, aryl, aralkyl, or C₃-C₆ cycloalkyl.

The (S)-pregabalin nitrile is prepared by a process comprising: a) combining a (±)-2-carboxyalkyl-3-cyano-5-methyl hexanoic acid ester of the following structure: 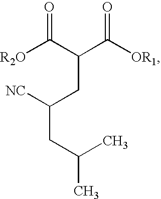
a solvent selected from the group consisting of water, a C₁-C₆ alcohol, and mixtures thereof, and an alkali metal base to obtain an alkaline salt of pregabalin nitrile of the following structure 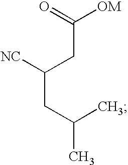
b) combining the above alkaline salt of pregabalin nitrile and an inorganic acid to obtain a mixture having pregabalin nitrile of the following structure 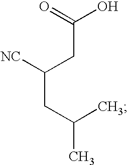
c) combining the above pregabalin nitrile, a solvent selected from the group consisting of ketones, esters, nitrites, C₁₋₄ alcohols, water, and mixtures thereof, and a chiral resolution reagent to obtain a precipitate of a diastereomeric salt; and d) combining the precipitated diastereomeric salt with an inorganic acid to obtain (S)-pregabalin nitrile, wherein M is an alkali metal and R₁ and R₂ are the same or different and are C₁-C₆ alkyl, aryl, aralkyl, or C₃-C₆ cycloalkyl. Preferably, the aryl is C₆-C₁₀ aryl. Preferably, the aralkyl is benzyl. Preferably, R₁ and R₂ are independently selected from C₁-C₆ alkyl, more preferably C₁-C₃ alkyl, and most preferably methyl or ethyl. Particularly preferred are compounds where RI and R₂ are both ethyl.

The starting (±)-2-carboxyalkyl-3-cyano-5-methyl hexanoic acid ester may be prepared according to the method described in the '767 patent, hereby incorporated by reference.

Typically, the chiral resolution reagent is a chiral amine resolution reagent. Preferably, the chiral amine resolution agent is selected from the group consisting of phenylethylamine, naphtylethylamine, D-glucamine, L-lysine, L-proline, brucine, sparteine, ephedrine, norephedrine, and salts thereof.

Preferably, the C₁-C₆ alcohol is a C₁-C₃ alcohol, and more preferably methanol or ethanol. Preferably, the solvent is selected from the group consisting of acetone, methyl iso-butyl ketone (“MIBK”), acetonitrile (“ACN”), methanol, ethanol, propanol, isopropyl alcohol (“IPA”), and butanol.

Preferably, the base is an alkaline hydroxide. Preferably, the alkaline hydroxide is selected from the group consisting of Ba(OH)₂, KOH, LiOH and NaOH. More preferably, the alkaline hydroxide is either KOH or NaOH.

Typically, the combination of (±)-2-carboxyalkyl-3-cyano-5-methyl hexanoic acid ester, solvent, and alkali metal base is stirred for about 2 hours to about 20 hours to obtain the alkaline salt of pregabalin nitrile. The alkaline salt of Pregabalin nitrile can be a racemic mixture of both enantiomers or a mixture of the enantiomers in any ratio.

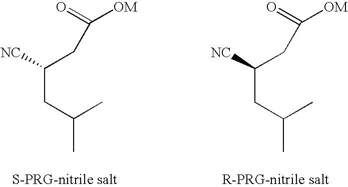

Preferably, the mixture is stirred at a temperature of about 20° C. to about 120° C., more preferably at a temperature of about 20° C. to about 25° C.

Preferably, the inorganic acid of step b) is selected from the group consisting of HBr, H₂SO₄, H₃PO₄, and HCl, and more preferably H₂SO₄ or HCl. Preferably, the inorganic acid is present in an amount sufficient to obtain a pH of about 2 to about 4, and more preferably about 4.

The obtained pregabalin nitrile may be isolated prior to step c). Preferably, the pregabalin nitrile is isolated by adding an organic solvent to the mixture to obtain a two phase system, separating the organic phase, and removing the organic solvent to obtain a residue of pregabalin nitrile. The obtained pregabalin nitrile can be a racemic mixture of both enantiomers or a mixture of the enantiomers in any ratio.

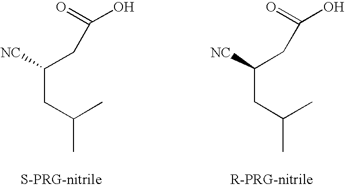

Preferably, the organic solvent of step c) is an ether or an ester. Preferably, the ether is a C₄-C₈ ether, and more preferably diethyl ether. Preferably, the ester is a C₂-C₈ ester, and more preferably ethyl acetate. Preferably, the organic solvent is removed by evaporation. The residue of pregabalin nitrile may optionally be purified by crystallization. Typically, the pregabalin nitrile is crystallized from isopropyl alcohol (“IPA”).

Typically, the combination of pregabalin nitrile, solvent and chiral resolution reagent is heated to obtain the diastereomeric mixture of the corresponding salt. Preferably, the combination is heated at a temperature of about 40° C. to about 140° C., and more preferably, at about the reflux temperature of the solvent. The diastereomeric mixture of the corresponding salt thus obtained is a mixture of the following diastereomers: 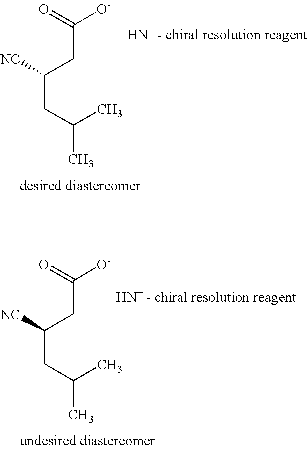
where the amine group of the chiral resolution reagent reacts with the carboxylic acid group of the pregabalin nitrile to form the salt.

The desired diastereomer of the salt is precipitated. Preferably, the combination is cooled to precipitate the desired diastereomer. Preferably, after heating, the combination is cooled at a temperature of about 0° C. to about 25° C., and more preferably at about 2° C. to precipitate the desired diastereomer, while the undesired diastereomer remains in solution.

In an alternative embodiment, the chiral resolution reagent may be chosen such that the undesired diastereomer of the salt is precipitated and the desired diastereomer of the salt remains in solution. The precipitated undesired diastereomer of the salt may then be removed and the desired diastereomer of the salt recovered from the solution by any method known to one of ordinary skill in the art.

The precipitated diastereomeric salt may be isolated prior to step d). Preferably, the precipitated diastereomeric salt is isolated by filtration.

Typically, the precipitated diastereomeric salt is dissolved in water prior to combining with the inorganic acid. Preferably, the precipitated diastereomeric salt and water are heated to form the solution. Preferably, the precipitated diastereomeric salt and water are heated at a temperature of about 50° C. to about 100° C. Preferably, the solution is then cooled to a temperature of about 20° C. to about 30° C., and more preferably to about room temperature. As used herein, “room temperature” means about 25° C.

After cooling, the inorganic acid is added to the solution. Preferably, the inorganic acid of step (d) is selected from the group consisting of HBr, H₂SO₄, H₃PO₄, and HCl. More preferably, the inorganic acid is HCl. Preferably, the solution containing the inorganic acid is further cooled at a temperature of about 25° C. to about 0° C., and, more preferably at a temperature of about 2° C. to obtain a precipitate of (S)-pregabalin nitrile. Preferably, the cooled solution is stirred for about 1 to about 24 hours.

The precipitated (S)-pregabalin nitrile may be recovered by any method known to one of ordinary skill in the art. Preferably, the precipitated (S)-pregabalin nitrile is recovered by filtration.

The (S)-pregabalin nitrile thus obtained may then be converted to (S)-pregablin. The conversion may be performed, for example, by the method disclosed in U.S. Pat. No. 5,637,767.

Having described the invention with reference to certain preferred embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Example 1

### Preparation of (3S)-Cyano-5-methylhexanoic acid

### a) Step 1: Hydrolysis and Decarboxylation of (±)-2-Carboxyethyl-3-cyano-5-methyl hexanoic acid ethyl ester

A reactor (0.5 l) is loaded with (±)-2-carboxyethyl-3-cyano-5-methyl hexanoic acid ethyl ester (50 g), and methanol (“MeOH”) (53 g). A solution of KOH (17.8 g) in water (56 ml) is added, while keeping the temperature below 25° C. The mixture is stirred for 2 hours at room temperature, and HCl is added to obtain pH 4. The solution is extracted with ethyl acetate (“EtOAc”) (2×50 ml), the organic phases are combined and concentrated. The product is crystallized from IPA.

### b) Step 2: Optical Resolution of 3-Cyano-5-methylhexanoic acid racemate

A 0.5 l flask is charged with acetone (320 ml), 3-cyano-5-methylhexanoic acid racemate (40 g), and S-phenyl ethyl amine (26 g). The mixture is heated to reflux, and stirred for 1 hour. The mixture is cooled to 2° C., and, after stirring for 1 hour at 2° C., the precipitate is filtered. Water (320 ml) is added to the wet solid, and the mixture is heated until dissolution. After cooling to room temperature, HCl-32% (10 ml) is added. The solution is cooled to 2° C., and stirred for 1 hour to obtain a precipitate containing (3S)-cyano-5-methylhexanoic acid. The presence of (3S)-cyano-5-methylhexanoic acid is detected by a chiral HPLC method.

### Example 2

### Hydrolysis and Decarboxylation of (±)-2-Carboxyethyl-3-cyano-5-methyl hexanoic acid ethyl ester

A three-neck-flask (50 ml) was charged with MeOH (3.5 ml), and (±)-2-carboxyethyl-3-cyano-5-methyl hexanoic acid ethyl ester (10 g). A solution of KOH in MeOH (2.12 g of KOH in 9.1 ml of MeOH) was added drop-wise over a period of 15 minutes. The mixture was heated to reflux for 4 hours and then cooled to room temperature. The solvent was evaporated under vacuum, and the product, (+)-3-cyano-5-methylhexanoic acid potassium salt, was obtained as white solid (9.71 g).

### Example 3

### hydrolysis and decarboxylation of (±)-2-Carboxyethyl-3-cyano-5-methyl hexanoic acid ethyl ester

A three-neck-flask (250 ml) was charged with water (100 ml), (±)-2-carboxyethyl-3-cyano-5-methyl hexanoic acid ethyl ester (5 g) and Ba(OH)₂ (9.26 g). The mixture was stirred for 15 hours at room temperature and then water was added (200 ml). The solution was acidified with H₂SO₄-66% (4.36 g) and filtered. The filtrate was evaporated under vacuum to obtain the product, (±)-3-cyano-5-methylhexanoic acid, as yellowish gummy solid.

### Example 4

### Preparation of (S)-Pregabalin: Example based upon U.S. Pat. No. 5.637.767 (col. 12, 1. 46 to col. 13, 1. 21) starting with (S)-3-cyano-5-methyl hexanoic acid, ethyl ester

An 800 l still is charged with (S)-3-cyano-5-methyl hexanoic acid, ethyl ester (50.1 kg, 273 mol) and ethyl alcohol 2B (53 kg). A solution of potassium hydroxide (17.8 kg, 317 mol) in water (56 l) is added controlling the addition rate to maintain the batch temperature below 25° C. The mixture is stirred at 20° to 25° C. for about 1.5 hours. The batch is transferred to a hydrogenator containing sponge nickel (15.0 kg, 50% water wet), followed by a rinse of ethyl alcohol 2B (27 kg). The mixture is treated with hydrogen at about 50 psi for about 19 hours (hydrogen uptake stopped).

The nickel is removed by filtration, and the filter cake is rinsed with a mixture of 39 kg ethyl alcohol 2B and 111 l of water. Glacial acetic acid (22.8 kg, 380 mol) is added to the filtrate, while maintaining the batch temperature at less than 40° C. The batch is heated to 70° to 75° C. to dissolve the solids. The batch is slowly cooled to 0° to 5° C. to crystallize the product.

The solid is collected on a centrifuge, and rinsed with 160 l isopropyl alcohol that is previously cooled to 0° to 5° C.

The damp solid is dried in a vacuum tray drier under vacuum at 35° to 45° C. (28 hours) to give (S)-3-aminomethyl-5-methylhexanoic acid.

We claim:

1. A process for preparing (3S)-cyano-5-methylhexanoic acid comprising:
a) combining
a (±)-2-carboxyalkyl-3-cyano-5-methyl hexanoic acid ester of the following structure, 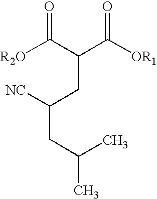
wherein R₁ and R₂ are the same or different and are C₁-C₆ alkyl, aryl, aralkyl, or C₃-C₆ cycloalkyl,
a solvent selected from the group consisting of water, a Cl -C₆ alcohol, and mixtures thereof, and
an alkali metal base
to obtain an alkaline salt of pregabalin nitrile of the following structure, 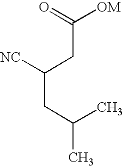
wherein M is an alkali metal;
b) combining the alkaline salt of pregabalin nitrile and an inorganic acid to obtain a mixture having (±)-3-cyano-5-methylhexanoic acid;
c) combining
the (±)-3-cyano-5-methylhexanoic acid,
a solvent selected from the group consisting of ketones, esters, nitriles, C₁₋₄ alcohols, water, and mixtures thereof, and
a chiral resolution reagent selected from the group consisting of phenylethylamine, naphtylethylamine, D-glucamine, L-lysine, L-proline, brucine, sparteine, ephedrine, norephedrine, and salts thereof
to obtain a precipitate of a diastereomeric salt; and
d) combining the precipitated diastereomeric salt with an inorganic acid to obtain (3S)-cyano-5-methylhexanoic acid.

2. The process of claim 1, wherein the C₁-C₆ alcohol is methanol or ethanol.

3. The process of claim 1, wherein the solvent is selected from the group consisting of acetone, methyl iso-butyl ketone, acetonitrile, methanol, ethanol, propanol, isopropyl alcohol, and butanol.

4. The process of claim 1, wherein the alkali metal base is an alkaline hydroxide.

5. The process of claim 4, wherein the alkali metal base is selected from the group consisting of Ba(OH)₂, KOH, LiOH and NaOH.

6. The process of claim 1, wherein the combination of (±)-2-carboxyalkyl-3-cyano-5-methyl hexanoic acid ester, solvent, and alkali metal base is stirred to obtain the alkaline salt of pregabalin nitrile.

7. The process of claim 6, wherein the combination of (±)-2-carboxyalkyl-3-cyano-5-methyl hexanoic acid ester, solvent, and alkali metal base is stirred at a temperature of about 20° C. to about 120° C.

8. The process of claim 1, wherein the inorganic acid of step b) is selected from the group consisting of HBr, H₂SO₄, H₃PO₄, and HCl.

9. The process of claim 1, wherein the inorganic acid of step b) is present in an amount sufficient to obtain a pH of about 2 to about 4.

10. The process of claim 1, wherein the combination of (±)-3-cyano-5-methylhexanoic acid, solvent, and chiral resolution reagent is heated to obtain a mixture having the diastereomeric salt.

11. The process of claim 10, wherein the combination is heated at a temperature of about 40° C. to about 140° C.

12. The process of claim 10, wherein the mixture having the diastereomeric salt is cooled to precipitate the diastereomeric salt.

13. The process of claim 12, wherein the mixture having the diastereomeric salt is cooled at a temperature of about 0° C. to about 25° C.

14. The process of claim 1, wherein the precipitated diastereomeric salt is dissolved in water prior to combining with the inorganic acid.

15. The process of claim 14, wherein the precipitated diastereomeric salt and water are heated to form the solution.

16. The process of claim 15, wherein the heating is to a temperature of about 50° C. to about 100° C.

17. The process of claim 15, wherein the heated solution is cooled to a temperature of about 25° C. to about 0° C., to obtain a precipitate of (S)-pregabalin nitrile.

18. The process of claim 1, wherein the inorganic acid of step d) is selected from the group consisting of HBr, H₂SO₄, H₃PO₄, and HCl.

19. A process for preparing (S)-pregabalin comprising:
a) preparing (3S)-cyano-5-methylhexanoic acid by the process of claim 1; and
b) converting the (3S)-cyano-5-methylhexanoic acid into (S)-pregabalin.

20. A process for optically resolving (3S)-cyano-5-methylhexanoic acid from (±)-3-cyano-5-methylhexanoic acid comprising:
a) combining
(±)-3-cyano-5-methylhexanoic acid,
a solvent selected from the group consisting of ketones, esters, nitrites, C₁₋₄ alcohols, water, and mixtures thereof, and
a chiral resolution reagent selected from the group consisting of phenylethylamine, naphtylethylamine, D-glucamine, L-lysine, L-proline, brucine, sparteine, ephedrine, norephedrine, and salts thereof to obtain a precipitate of a diastereomeric salt; and
b) combining the precipitated diastereomeric salt with an inorganic acid to obtain (3S)-cyano-5-methylhexanoic acid.

21. The process of claim 20, wherein the combination in step a) is heated at a temperature of about 40° C. to about 140° C. to obtain a solution.

22. The process of claim 21, wherein the combination having the diastereomeric salt is cooled to precipitate the diastereomeric salt.

23. The process of claim 22, wherein the combination having the diastereomeric salt is cooled at a temperature of about 0° C. to about 25° C.

24. The process of claim 20, wherein the precipitated diastereomeric salt is dissolved in water prior to combining with the inorganic acid.

25. The process of claim 24, wherein the precipitated diastereomeric salt and water are heated to form the solution.

26. The process of claim 25, wherein the heating is to a temperature of about 50° C. to about 100° C.

27. The process of claim 20, wherein the inorganic acid is selected from the group consisting of HBr, H₂SO₄, H₃PO₄, and HCl.

28. A process for preparing (S)-pregabalin comprising:
a) preparing (3S)-cyano-5-methylhexanoic acid by the process of claim 20; and
b) converting the (3S)-cyano-5-methylhexanoic acid into (S)-pregabalin.

29. A process for preparing (3S)-cyano-5-methylhexanoic acid comprising:
a) providing (±)-3-cyano-5-methylhexanoic acid; and
b) resolving (3S)-cyano-5-methylhexanoic acid from the (±)-3-cyano-5-methylhexanoic acid with a chiral resolution reagent selected from the group consisting of phenylethylamine, naphtylethylamine, D-glucamine, L-lysine, L-proline, brucine, sparteine, ephedrine, norephedrine, and salts thereof; and
c) adding an inorganic acid to obtain the (3S)-cyano-5-methylhexanoic acid.

30. A process for preparing (3S)-cyano-5-methylhexanoic acid comprising:
a) providing (±)-3-cyano-5-methylhexanoic acid; and
b) resolving (3S)-cyano-5-methylhexanoic acid from the (±)-3-cyano-5-methylhexanoic acid with a chiral resolution reagent.
